(19) United States
(12) Patent Application Publication
(10) Pub. No.: US 2023/0060934 A1
(43) Pub. Date: Mar. 2, 2023

(54) BOTANICAL ANTIVIRAL NUTRITIONAL SUPPLEMENT

(71) Applicant: Michael Wayne Dockery, Brockton, MA (US)

(72) Inventor: Michael Wayne Dockery, Brockton, MA (US)

(21) Appl. No.: 17/819,705

(22) Filed: Aug. 15, 2022

Related U.S. Application Data
(60) Provisional application No. 63/260,561, filed on Aug. 25, 2021.

(51) Int. Cl. A61K 36/80; A61K 36/28; A61K 36/41; A61K 36/185; A61K 36/746; A61K 36/536; A61K 36/537

(52) U.S. Cl. CPC A61K 36/80; A61K 36/28; A61K 36/41; A61K 36/185; A61K 36/746; A61K 36/536; A61K 36/537; A61K 2236/17; A61K 2236/15

(57) ABSTRACT

A botanical antiviral nutritional supplement includes ground Achillea Millefolium, ground Bryophyllum Pinnatum, ground Sambucus Nigra, ground Neurolaena Lobata, ground Verbascum Thapsus, ground Morinda Citrifolia, ground Prunella Vulgaris, and ground Salvia Officinalis. A method of producing the antiviral nutritional supplement includes drying the botanical ingredients; grinding the dried ingredients; and blending the ground, dried ingredients. The supplement is believed to improve the health of a person infected with a virus and to relieve brain fog.

### CROSS-REFERENCE TO RELATED APPLICATION

This application claims the benefit of priority of U.S. provisional application No. 63/260,561, filed Aug. 25, 2021, the contents of which are herein incorporated by reference.

### BACKGROUND OF THE INVENTION

The present invention relates to botanical remedies and, more particularly, to a botanical antiviral nutritional supplement.

Sometimes an individual gets sick from cold or flu viruses and needs a way to heal themselves and their immune system. Currently available herbal remedies do not have sufficient potency to relieve sickness in a hurry.

As can be seen, there is a need for a potent, fast-acting botanical remedy for viral infections.

### SUMMARY OF THE INVENTION

In one aspect of the present invention, a botanical antiviral nutritional supplement comprises ground Achillea Millefolium, ground Bryophyllum Pinnatum, ground Sambucus Nigra, ground Neurolaena Lobata, ground Verbascum Thapsus, ground Morinda Citrifolia, ground Prunella Vulgaris, and ground Salvia Officinalis.

In another aspect of the present invention, a method of producing an antiviral nutritional supplement comprises drying Achillea Millefolium, Bryophyllum Pinnatum, Sambucus Nigra, Neurolaena Lobata, Verbascum Thapsus, Morinda Citrifolia, Prunella Vulgaris, and Salvia Officinalis; grinding the dried Achillea Millefolium, Bryophyllum Pinnatum, Sambucus Nigra, Neurolaena Lobata, Verbascum Thapsus, Morinda Citrifolia, Prunella Vulgaris, and Salvia Officinalis; and blending the ground, dried Achillea Millefolium, Bryophyllum Pinnatum, Sambucus Nigra, Neurolaena Lobata, Verbascum Thapsus, Morinda Citrifolia, Prunella Vulgaris, and Salvia Officinalis.

The present invention provides a unique combination of fruits and herbs that boosts and replenishes the immune system and body. The inventive composition is believed to improve the health of a person infected with a virus and to relieve brain fog.

These and other features, aspects and advantages of the present invention will become better understood with reference to the following drawings, description, and claims.

### BRIEF DESCRIPTION OF THE DRAWINGS

FIG. 1 is a schematic of a method of manufacturing a botanical antiviral nutritional supplement according to an embodiment of the present invention;

FIG. 2 is a schematic view showing formulations of a botanical antiviral nutritional supplement according to an embodiment of the present invention; and

FIG. 3 is a perspective view of a package thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a botanical illness remedy comprising Achillea Millefolium, Bryophyllum Pinnatum, Sambucus Nigra, Neurolaena Lobata, Verbascum Thapsus, Morinda Citrifolia, Prunella Vulgaris, and Salvia Officinalis. Each component individually is effective at fighting some viruses and pathogens but together they perform surprisingly well in destroying the disease and rebuilding the body's immune system.

Achillea millefolium, also known as Yarrow, has been used in traditional medicine for treating cuts and abrasions, as an analgesic, and as a head cold remedy.

Bryophyllum Pinnatum, or Leaf of Life, has been used as a traditional treatment for hypertension, kidney stones, headaches, inflammation, cancer, and as a remedy for fevers.

Extracts of the Elderberry flower and fruit, also known as Sambucus Nigra, is a common dietary supplement and has been used in traditional medicine for cold and flu symptoms.

Some studies have suggested that Neurolaena Lobata, also known as “Jackass Bitters” or Zebapique, may have anti-inflammatory and antimicrobial properties. Zebapique has also been used in folk medicine to treat a variety of ailments.

Mullein, or Verbascum thapsus, has commonly been used in traditional medicine, for example for pulmonary diseases.

Noni, also known as Morinda Citrifolia, has been used as a dietary supplement.

Prunella Vulgaris, or Prunella, has been used in traditional medicine to heal wounds and throat infections, and may have additional health-related properties. For example, Prunella may help prevent diabetes complications, may have cancer-fighting properties, may help treat herpes, and may have anti-inflammatory properties.

Sage, also referred to as Salvia Officinalis, has a long history of use for its healing properties. Extracts of sage are undergoing research for effects on human brain function.

In some embodiments, Achillea Millefolium, Bryophyllum Pinnatum, Sambucus Nigra, Neurolaena Lobata, Verbascum Thapsus, Morinda Citrifolia, Prunella Vulgaris, and Salvia Officinalis may all be provided in equal amounts. The ratios may be optimized to help a person's immune system depending on the ailments the person has, such as for lung treatment versus for liver treatment. For example, Prunella, or Elderberry, and/or Mullein may be provided in a weight ratio of from about 1:1 to about 4:1 as compared to each of the remaining ingredients. In some cases, the total dry matter of the composition may be about 8 parts by weight to about 17 parts by weight. In some embodiments, the dry ingredients are present in equal amounts.

In some embodiments, the composition consists essentially of Achillea Millefolium, Bryophyllum Pinnatum, Sambucus Nigra, Neurolaena Lobata, Verbascum Thapsus, Morinda Citrifolia, Prunella Vulgaris, and Salvia Officinalis. The transitional phrase “consisting essentially of” limits the scope of a claim to the specified materials or steps “and those that do not materially affect the basic and novel characteristic(s)” of the claimed invention. In this case, the basic and novel characteristics include a botanical composition (i.e., nonbotanical components other than water may be excluded) that is effective to boost a user's immune system and help prevent or remedy a viral infection.

To make the botanical blend, the leaf and/or fruit ingredients may be dried and ground, e.g., in a blender, into powder form.

A user may consume the powdered composition in the form of a hot tea, a capsule, a pill, or a cold drink to help boost their immune system.

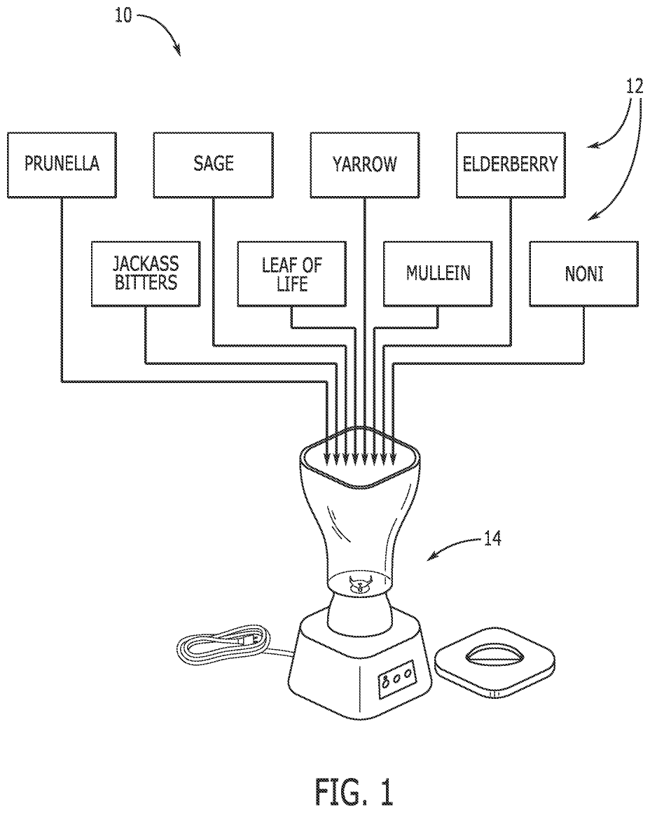

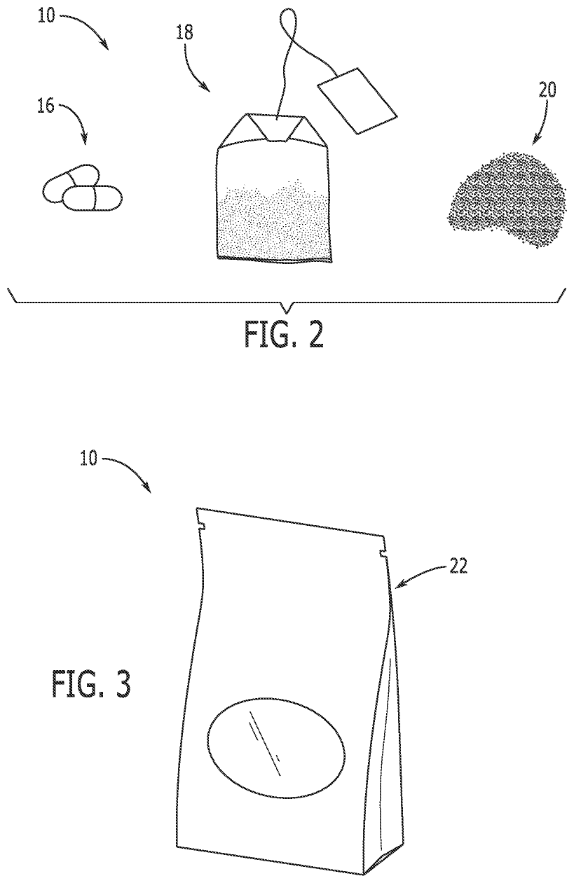

Referring to FIGS. 1 through 3, FIG. 1 illustrates a method of producing a botanical antiviral remedy or nutritional supplement 10. The eight ingredients 12 may be dried and ground in a blender or mixing unit 14 to produce a blended, powdered nutritional supplement according to an embodiment of the present invention for fighting viral infections. As shown in FIG. 2, the botanical antiviral nutritional supplement 10 may be formulated as a pill 16, a packaged tea 18, or a powder 20 and may be packaged as a packaged product 22, as illustrated in FIG. 3.

It should be understood, of course, that the foregoing relates to exemplary embodiments of the invention and that modifications may be made without departing from the spirit and scope of the invention as set forth in the following claims.

What is claimed is:

1. A botanical antiviral nutritional supplement comprising: ground Achillea Millefolium, ground Bryophyllum Pinnatum, ground Sambucus Nigra, ground Neurolaena Lobata, ground Verbascum Thapsus, ground Morinda Citrifolia, ground Prunella Vulgaris, and ground Salvia Officinalis.

2. The botanical antiviral nutritional supplement of claim 1, wherein:
the ground Achillea Millefolium is present in an amount of about 1 part by weight;
the ground Bryophyllum Pinnatum is present in an amount of about 1 part by weight;
the ground Sambucus Nigra is present in an amount of about 1 part by weight to about 4 parts by weight;
the ground Neurolaena Lobata is present in an amount of about 1 part by weight;
the ground Verbascum Thapsus is present in an amount of about 1 part by weight to about 4 parts by weight;
the ground Morinda Citrifolia is present in an amount of about 1 part by weight;
the ground Prunella Vulgaris is present in an amount of about 1 part by weight to about 4 parts by weight; and
the ground Salvia Officinalis is present in an amount of about 1 part by weight;
wherein the botanical antiviral nutritional supplement has a total dry matter of about 8 parts by weight to about 17 parts by weight.

3. The botanical antiviral nutritional supplement of claim 1, wherein the ground Achillea Millefolium, the ground Bryophyllum Pinnatum, the ground Sambucus Nigra, the ground Neurolaena Lobata, the ground Verbascum Thapsus, the ground Morinda Citrifolia, the ground Prunella Vulgaris, and the ground Salvia Officinalis are present in equal amounts.

4. The botanical antiviral nutritional supplement of claim 1, wherein the ground Achillea Millefolium, the ground Bryophyllum Pinnatum, the ground Sambucus Nigra, the ground Neurolaena Lobata, the ground Verbascum Thapsus, the ground Morinda Citrifolia, the ground Prunella Vulgaris, and the ground Salvia Officinalis are dried.

5. The botanical antiviral nutritional supplement of claim 1, formulated as a hot tea, a capsule, a pill, or a cold drink.

6. A botanical antiviral nutritional supplement consisting essentially of: ground Achillea Millefolium, ground Bryophyllum Pinnatum, ground Sambucus Nigra, ground Neurolaena Lobata, ground Verbascum Thapsus, ground Morinda Citrifolia, ground Prunella Vulgaris, and ground Salvia Officinalis.

7. A method of producing an antiviral nutritional supplement comprising:
drying Achillea Millefolium, Bryophyllum Pinnatum, Sambucus Nigra, Neurolaena Lobata, Verbascum Thapsus, Morinda Citrifolia, Prunella Vulgaris, and Salvia Officinalis;
grinding the dried Achillea Millefolium, Bryophyllum Pinnatum, Sambucus Nigra, Neurolaena Lobata, Verbascum Thapsus, Morinda Citrifolia, Prunella Vulgaris, and Salvia Officinalis; and
blending the ground, dried Achillea Millefolium, Bryophyllum Pinnatum, Sambucus Nigra, Neurolaena Lobata, Verbascum Thapsus, Morinda Citrifolia, Prunella Vulgaris, and Salvia Officinalis.

8. The method of claim 7, wherein the ground, dried Achillea Millefolium, Bryophyllum Pinnatum, Sambucus Nigra, Neurolaena Lobata, Verbascum Thapsus, Morinda Citrifolia, Prunella Vulgaris, and Salvia Officinalis are blended in equal amounts.

9. The method of claim 7, further comprising formulating the blended, ground, dried Achillea Millefolium, Bryophyllum Pinnatum, Sambucus Nigra, Neurolaena Lobata, Verbascum Thapsus, Morinda Citrifolia, Prunella Vulgaris, and Salvia Officinalis as a hot tea, a capsule, a pill, or a cold drink.
